(19) United States
(12) Patent Application Publication
(10) Pub. No.: US 2023/0032290 A1
(43) Pub. Date: Feb. 2, 2023

(54) IMMERSION ASSESSMENT SYSTEM AND ASSOCIATED METHODS

(71) Applicant: Immersion Neuroscience, Inc., Henderson, NV (US)

(72) Inventors: Jorge A. Barraza, Claremont, CA (US); Paul J. Zak, Loma Linda, CA (US)

(73) Assignee: Immersion Neuroscience, Inc., Henderson, NV (US)

(21) Appl. No.: 17/874,114

(22) Filed: Jul. 26, 2022

Related U.S. Application Data
(60) Provisional application No. 63/227,544, filed on Jul. 30, 2021.

(51) Int. Cl. G06Q 30/02

(52) U.S. Cl. CPC G06Q 30/0203

(57) ABSTRACT

An immersion assessment system for assessing immersion levels of one or more experience participants based on heart rhythm data collected from one or more participants during an experience is described. The system includes an ingestion data hub for processing heart rhythm data to provide clean data, and a neuroscience processing unit for analyzing the clean data and providing analytical results including primary metrics. The system further includes a behavior analysis unit for further analyzing the clean data and the analytical results to provide secondary metrics, and a workflow management unit for controlling the ingestion data hub, the neuroscience processing unit, and the behavior analysis unit. The system may further include a content control unit for presenting the experience to one or more experience participants and correlating the analytical results with specific parameters and timing associated with the experience.

### FIELD OF THE INVENTION

The present invention relates to methods and systems for assessing participant engagement with presented content and experiences and, more specifically, the systems and methods for assessing neurologic immersion of one or more participants with presented content and experiences based on simultaneous measurement of physiologic data, optionally from multiple people.

### BACKGROUND OF THE INVENTION

Today, businesses make decisions based on what people “feel” or what they “like” using surveys, focus groups, or an executive's “intuition.” Science and history have shown that these decisions are only right roughly 17% of the time.

A variety of methods have been used to more rigorously measure people's engagement with a particular experience, such as an advertisement, media content, or live experience, and predicting participant behavior, although these methods have disadvantages in practical usage. Such methods include the following examples.

1) Eye-tracking: This method measures visual attention by using sensors to track the movement of a participant's eyes. However, the emotional impact or value of content on the participant cannot be measured. There is usually a high rate of data loss (e.g., 50% or more), especially with remote eye-tracking solutions, due to stringent lighting and head orientation requirements.

2) Automated facial coding: This method involves capturing a person's facial muscle movements using a camera while the participant is presented with media content, such as video clips on a computer. Popularized by the work of psychologist Paul Ekman, facial coding is one of the most widely utilized measures in neuromarketing as the data capture is simple and the data analysis can be automated using algorithms. However, academic research has shown that the facial coding method is poor at capturing emotions accurately (see, for example, L. F. Barrett, et al., “Emotional Expressions Reconsidered: Challenges to Inferring Emotion from Human Facial Movements,” Psychological Science in the Public Interest, vol. 20, Issue 1, pp. 1-68, Jul. 17, 2019 (https://journals.sagepub.com/doi/full/10.1177/1529100619832930 accessed Jul. 7, 2021)). Moreover, the technology is almost entirely focused on presenting content via a computer in a structured environment with sufficient lighting, where the participant is asked to remain still and keep their head in one position within a few feet of the camera.

3) Electroencephalogram (EEG): EEG devices use electrodes that are attached to specific locations on a participant's scalp to detect electrical activity in the participant's brain. There is a high variance in the quality of EEG devices. For instance, devices with only a few electrodes, while easy to use, are often unreliable and inaccurate in their readouts compared to medical-grade EEGs. On the other hand, medical grade EEG caps are cumbersome, uncomfortable to wear, and can only be used in a lab setting. Furthermore, correspondence between EEG data and specific emotions has not been solidly and scientifically established. The use of EEG devices is generally cost-prohibitive to scale for use in realistic experiences people have and is nearly impossible for multi-participant situations.

4) Galvanic Skin Response (GSR): GSR devices detect changes in sweat gland activity, which lead to changes in electrical properties of the skin measurable as, for instance, skin conductance. It is difficult to ensure accuracy and fidelity of GSR data, as they are highly dependent on the collection environment (e.g., variations in skin physiology, external temperatures in the 68 to 72 Fahrenheit range, etc.) and are incredibly sensitive to normal movement.

5) Implicit reaction time: Rooted in academic research on racial and gender biases, this analysis supposes that the reaction time (i.e., speed of response) of participants to specific stimuli is shortened when the brain is more strongly engaged in the activity. However, neither reliability nor predictive validity has been scientifically established for the correlation between implicit reaction time and real-world behaviors.

The aforementioned and other existing methods have disadvantages for practical usage in terms of hardware costs, effort, expertise, sensitivity, and accuracy. Accordingly, a system and method for accurately assessing neurologic responses and predicting associated behavior of participants in a given experience would be desirable.

### SUMMARY OF THE INVENTION

In accordance with the embodiments described herein, there is described a neurologic immersion assessment system for assessing immersion levels of one or more experience participants based on heart rhythm data collected from the one or more people during an experience. The system includes an ingestion data hub for processing the heart rhythm data to provide clean data, and a neuroscience processing unit for analyzing the clean data and providing analysis results including primary metrics. The system further includes a behavior analysis unit for further analyzing the clean data and the analysis results to provide secondary metrics, and a workflow management unit for controlling the ingestion data hub, the neuroscience processing unit, and the behavior analysis unit. As used herein, clean data includes heart rhythm time series data that have been processed, for example, by removing illogical information (e.g., heart rate above or below specified thresholds), aligning incoming data from multiple experience participants with timing specific for a specific event, and/or calibrating the incoming data according to sensor type.

In accordance with a further embodiment, the immersion assessment system includes a content control unit, interfaced with at least the neuroscience processing unit, for presenting the experience to the one or more experience participants and correlating the analysis results with specific parameters and timing associated with the experience.

### BRIEF DESCRIPTION OF THE DRAWINGS

FIG. 1 shows a block diagram of a system for assessing the immersion level of one or more participants with presented content and experiences and predicting experience participant behavior, in accordance with an embodiment.

FIG. 2 is a block diagram of a computing system for assessing the immersion level of one or more participants, in accordance with an embodiment.

FIG. 3 shows a flow diagram for using a system for assessing the immersion level of one or more participants with presented content and experiences, in accordance with an embodiment.

FIG. 4 shows an exemplary graph of processed heart rhythm or cardiac data as measured, along with an illustration of exemplary steps in analyzing the data presented in the graph, in accordance with an embodiment.

FIG. 5 shows a flow diagram for performing the steps corresponding to the illustration in FIG. 4, in accordance with an embodiment.

### DETAILED DESCRIPTION OF THE EMBODIMENTS

The present invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity. Like numbers refer to like elements throughout.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items, and may be abbreviated as “/”.

It will be understood that when an element or layer is referred to as being “on,” “connected to,” “coupled to,” or “adjacent to” another element or layer, it can be directly on, connected, coupled, or adjacent to the other element or layer, or intervening elements or layers may be present. In contrast, when an element is referred to as being “directly on,” “directly connected to,” “directly coupled to,” or “immediately adjacent to” another element or layer, there are no intervening elements or layers present. Likewise, when light is received or provided “from” one element, it can be received or provided directly from that element or from an intervening element. On the other hand, when light is received or provided “directly from” one element, there are no intervening elements present.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

Knowing what people's brains value is imperative for creating a transformative experience and has led to the proliferation of methods for assessing engagement using surveys, tests, and biometric measurements such as discussed above. Today, companies use such methodologies, sometimes referred to as neuromarketing, consumer neuroscience, or applied neuroscience, in fields such as advertising, marketing, training, and entertainment.

Rigorous neuroscience research in the past several decades has established a relationship between what a person is experiencing and the corresponding neurochemicals produced by that person's brain. In particular, the secretion of neurochemicals oxytocin and dopamine have been established as key signals showing that the brain values an experience. For instance, researchers have found connections between the presence of oxytocin and social behaviors such as trustworthiness, generosity, charitable giving (See, for example, 1) Zak, Stanton, Ahmadi, 2007; 3) Zak, Kurzban, Matzner, 2005; 3) Barraza, Zak, 2009; 4) Barraza, McCullough, Ahmadi, Zak, 2011; and 5) Lin, Gerwal, Morin, Johnson, Zak 2013), purchases (Alexander, Tripp & Zak, 2015) and collective action (Zak & Barraza 2013)).

Physiologically, the presence of oxytocin has been shown to correspondingly modulate the heart's rhythms in measurable ways (See, for example, 1) Porges, 2001; 2) Thayer, Lane, 2009; 3) Kemp, Quintana, et al., 2012; 4) Norman, Cacioppo, et al, 2011; 5) Barraza, Terris, et al., 2015; 6) Jurek, Neumann, 2018; 7) Gutkowska, Jakowski, 2012). As an example, the presence of oxytocin affects the level of adrenocorticotropic hormone (ACTH) in a person's blood stream, which in turn produces changes in the person's heart rhythm. Consequently, by monitoring subtle changes in heart rhythms, the brain's neurochemical response to an experience can be inferred such that heart rhythm data, such as collected using photoplethysmography (PPG), can be used to assess the person's reaction to an experience.

For instance, if a person is emotionally resonating with an experience, e.g., watching a movie or a commercial, sitting in a class, or working with a team, that person's brain typically releases oxytocin both into the brain and via the pituitary gland into the bloodstream. As oxytocin is simultaneously released into the brain and the bloodstream, a change in the oxytocin level in the blood generally reflects the activity of oxytocin in the brain. In the bloodstream, oxytocin binds to the vagus nerve and heart, thereby subtly changing the heart's rhythms (Norman et al., 2011). Thus, measurement of changes in heart rhythms can be used to infer the person's engagement with an experience at a particular moment in time.

An indicator of such a state of engagement is “immersion.” Immersion is defined as a biological state of attention and emotional resonance in the brain, measurable by changes in the balance of neurochemicals in the blood stream. Due to the effects of these neurochemical changes on the peripheral nervous system, a person's level of immersion can also be inferred by monitoring subtle changes in the person's heart rhythms, as established in scientific research cited above. For instance, analysis of immersion has been shown to predict what people will do and remember after an experience with over 80% accuracy.

In other words: 1) Immersion is a neurologic state of attention and emotional resonance with an experience; and 2) The state of immersion is predictive of experience outcomes. For instance, if immersion is high for an advertisement, that ad will be better remembered by a consumer and will predispose the consumer to take action (e.g., purchase, share on social media).

Due to recent advances in PPG sensors found in many common wearable devices, immersion levels can be assessed using commercial wearable devices as well as built-in smartphone cameras. Therefore, a system for simultaneously assessing immersion levels of multiple people using heart rhythm data via PPG sensing is described herein. That is, using PPG sensors that are widely available in smartwatches and fitness trackers, changes in patterns in heart rate, and the neurochemistry changes associated therewith, may be analyzed to simultaneously assess immersion levels of a large number of people outside of a laboratory environment. Included are also other sensing devices that enable obtaining heart rhythm data, such as built-in cameras on smartphones that utilize finger contact over the camera lens (see Coppetti, et al., 2017). The relevant heart rhythm data may include, for example, heart rate, heart rate variability, pulse rate variation, and other heart activity information.

As described herein, an immersion assessment system enables simultaneous heart rhythm data capture and assessment for one or more participants, along with a variety of interfaces (e.g., mobile, web, and desktop applications) to provide feedback to stakeholders for reporting and workflow management. For instance, the immersion assessment system of the present disclosure enables simultaneous evaluation of immersion levels of multiple participants' experience synchronously or asynchronously, thus providing accurate behavioral prediction.

It is noted that, within the present disclosure, the term “experience” may cover, for instance, pre-recorded media (such as entertainment content, training sessions, and educational videos), market research scenarios (e.g., staged settings with controlled variables like product experiences), and live events. That is, within the present disclosure, the participants in the various experiences encompass more than passive audiences, and experience participants may be actively engaged with presented scenarios, such as a mock shopping experience, a rock concert, or a live seminar.

In an embodiment, the immersion assessment system includes a distributed neuroscience software platform for collecting data from smartwatches or fitness sensors of multiple experience participants to directly measure, second by second, what an experience participant's brain values, enabling real-time, moment-by-moment, assessment of the experience participants' immersion levels, collected simultaneously from multiple experience participants. The assessments may be aggregated to provide additional insights in situations that would not be possible in a controlled, laboratory environment. That is, unlike previous engagement analysis systems that are limited to data collection from one or a few experience participants at a time within a confined setting such as an observation room or a laboratory, the immersion assessment system of the present disclosure enables near real-time collection and viewing of data from a plurality of viewers of specific media content, or even attendees of live events such as educational seminars, for nearly any type of experience in a non-obtrusive way using commonly-used PPG data collection wearables such as smartwatches and fitness trackers, or using PPG approaches using a built-in camera of a smart device, such as fingertip contact photoplethysmography (e.g., measuring finger pulse by contacting a fingertip to a built-in camera of a smart device) or non-contact photoplethysmography (e.g., using the built-in camera of a smart device to measure heart rhythm data). Heart rhythm measurement may be performed by approaches other than PPG, as long as the heart rhythm data can be collected with sufficient accuracy and resolution to enable performance of the analytic processes described below.

More particularly, the immersion assessment system of the present disclosure uses heart rhythm data to assess two key indicators of neurologic immersion, namely: 1) attention to the experience; and 2) emotional resonance. As a person's attention increases during an experience, the activity in the person's brain's prefrontal cortex causes an increase in sympathetic activity measurable from cardiac (or equivalently, heart rhythm) data. Also, as discussed above, emotional resonance is associated with the brain's synthesis of the neurochemical oxytocin, which increases activity of the vagus nerve, thus altering the person's heart rhythm in detectable ways. The immersion assessment system of the present disclosure quantifies the neurologic response to a given experience by measuring changes in the heart rhythm and analyzing the measurements for corresponding indication of brain activity. Taking real-time heart rhythm data from one or simultaneously from a plurality of individuals sharing an experience, then processing the data using measured changes in heart rhythms, the immersion assessment system of the present disclosure enables simultaneous assessment of the immersion level of a plurality of experience participants essentially in real-time.

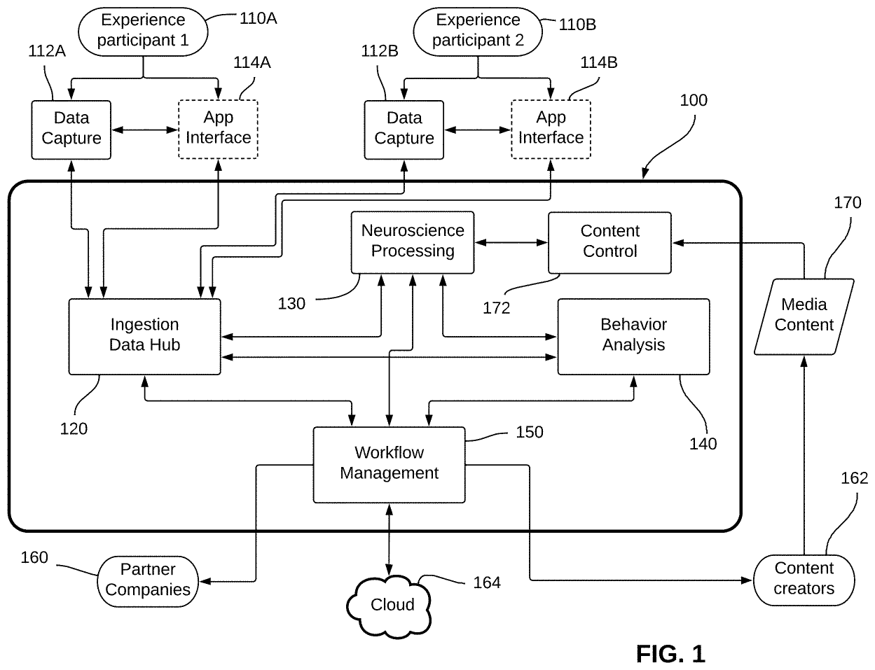

Turning now to the figures, FIG. 1 shows a block diagram of a system for assessing the level of immersion with an experience and predicting behavior of one or more experience participants, in accordance with an embodiment. As shown in FIG. 1, an immersion assessment system 100 interfaces with one or more experience participants (shown as 110A and 110B) through a data capture mechanism 112A and 112B, respectively. Experience participants 110A and B may be, for example, test participants being shown a film clip, a participant at a seminar, a movie goer, or an event attendee. It is noted that, while only two bubbles representing experience participants 110A and 110B are shown in FIG. 1, data capture may be simultaneously performed for just one participant, two or more experience participants, who may be simultaneously involved in the same experience, in the same experience at staggered times, or in different experiences at the same time.

Data capture mechanism 112A and 112B may be a device capable of capturing real-time heart rhythm data of the respective experience participant. As an example, data capture mechanism is a smartwatch or a fitness tracker worn by the experience participant to capture real-time heart rhythm data of experience participant. Alternatively, the experience participant may be directed to use the PPG capture feature of a smart device (e.g., the camera of a smart phone) while participating in the experience. While only two experience participants 110A and 110B are shown in FIG. 1, immersion assessment system 100 may be interfaced with just one experience participant or a plurality of experience participant members, with each experience participant associated with their own data capture mechanism (e.g., a smartwatch or fitness tracker worn by that experience participant). The heart rhythm data of the one or more experience participants may be transmitted to immersion assessment system 100 via a wired or wireless (e.g., Bluetooth® connection or other) connectivity mechanism in real-time or some time post experience.

Optionally, each experience participant may interact with an application interface (e.g., 114A and 114B as shown in FIG. 1) on a mobile device or a computer. Application interface may include, for example, a mobile application configured for communicating with immersion assessment system 100 and providing an interactive user interface for each experience participant. For instance, the application interface may display the experience to be assessed (e.g., media content, advertisement, event recording, or live event), provide an interface for each experience participant to adjust user settings, monitor the data capture mechanism, and/or send and receive information from immersion analysis system 100. Further, immersion analysis system 100 may be configured for accommodating a variety of data capture mechanisms and application interfaces (e.g., a Fitbit® fitness tracker connected via an iOS® operation system application as well as a Garmin® fitness tracker connected via an Android® operating system)

Continuing to refer to FIG. 1, immersion analysis system 100 includes an ingestion data hub 120 for interfacing with the experience participant(s) via data capture mechanism and/or application interface. Ingestion data hub 120 performs a variety of tasks such as pairing data from a specific experience participant with a specific event to be analyzed, clean the incoming heart rhythm data to remove illogical information (e.g., heart rate above or below specified thresholds), align incoming data from multiple experience participants with timing specific for a specific event, and calibrate the incoming data according to sensor type. Ingestion data hub 120 thus receives and processes the incoming heart rhythm data from one or more experience participants to provide clean data.

Immersion analysis system 100 also includes a neuroscience processing unit 130. Neuroscience processing unit 130 analyzes the clean data from ingestion data hub 120 to generate analytical results, such as primary metrics such as immersion and psychological safety by correlating received heart rhythm data with established neurochemical analyses, such as described above. Neuroscience processing unit 130 may also perform analyses such as the identification of key moments within the experience being analyzed, and the grouping of the experience timeline into time periods of high or low immersion. As an example, the grouping of the experience timeline into time periods of high or low immersion may be performed using a process referred to herein as “pilling,” as will be described in further detail below.

In the exemplary embodiment shown in FIG. 1, the immersion assessment system 100 further includes a behavior analysis unit 140. Behavior analysis unit 140 may receive the clean data from ingestion data hub 120 and the analysis results from neuroscience processing unit 130 to perform further analyses such as, for instance, aggregate profiling, vertical analysis, and pattern analysis. Optionally, neuroscience processing unit 130 and/or behavior analysis unit 140 may perform additional functions such as the calculation of secondary metrics (e.g., comparison of the primary metrics with established norms), identifying and clipping key moments in the experience, and generating summary reports (e.g., norm comparison, key moments (high and low points), participant breakdown, correlating key moments with specific points in the experience agenda, generating annotated video of the experience with immersion assessment results). The primary and/or secondary metrics may optionally be sent via ingestion data hub 120 to be displayed to experience participant 110 via, for example, application interface 114.

Immersion assessment system 100 of FIG. 1 further includes a workflow management unit 150. Workflow management unit 150 may include, for example, interfaces with ingestion data hub 120, neuroscience processing unit 130, and/or behavior analysis unit 140 for receiving and aggregating data from each of these system components. Workflow management unit 150 may also provide an interface between immersion assessment system 100 with external stakeholders, such as partner companies 160, who are users or clients of the immersion assessment system 100 or content creators 162, or provide aggregated data or analysis history to a cloud server 164. As an example, content creators 162 may include companies or personnel who produce the experience (e.g., event or media content 170) being assessed by the immersion assessment system 100. As another example, content creators may include content (or experience) participants who are managing the content/experience using the immersion assessment system 100 to organize the content/experience, invite selected experience participants to participate, and execute the measurement. Workflow management unit 150 may include a website or user interface for displaying, for instance, details related to experience participants 110 and media content 170, creation and management of experiences to be assessed, as well as data and analysis results visualization in real-time during the experience and/or after the conclusion of the experience. It is noted that media content 170 may be, for instance, a video recording of a live experience, or pre-recorded content presented to one or more experience participants.

In an example, media content 170 is provided by content creators 162 to a content control unit 172 for use in presenting the experience to be assessed (e.g., audiovisual content or online event) to experience participant 110 and in correlating the analysis results of neuroscience processing unit 130 with specific event timing of media content 170. Furthermore, content control unit 172 may provide media management functions to enable secure streaming of media content 170 to specific experience participants 110, or even adjust the content provided to each experience participant 110 according to the real-time analysis results from neuroscience processing unit 130.

It is noted that, while content control unit 172 is shown in FIG. 1 as being interfaced with neuroscience processing unit 130, content control unit 172 may be additionally or alternatively interfaced with ingestion data hub 120, behavior analysis unit 140, and/or workflow management unit 150. It is further noted that, while ingestion data hub 120, neuroscience processing 130, behavior analysis unit 140, workflow management unit 150, and content control unit 172 are shown as distinct components within the immersion assessment system 100, two or more of these components may be combined in a single unit.

It is further noted that, immersion assessment system 100 may be contained in a specialized hardware system integrating the various components therein, or implemented within a standalone computing system, including a processor and memory with programming executable by the processor to perform the functions of ingestion data hub 120, neuroscience processing unit 130, behavior analysis unit 140, workflow management unit 150, and content control unit 172. Alternatively, certain aspects of ingestion data hub 120, neuroscience processing unit 130, behavior analysis unit 140, workflow management unit 150, and/or content control unit 172 may be performed by dedicated hardware or within cloud 164. For instance, by providing certain aspects of the components within immersion assessment system 100 within cloud 164, specific functionalities of immersion assessment system 100 may be provided in a Software-as-a-Service configuration.

The immersion assessment system of the present disclosure differs from existing engagement assessment systems in at least the following ways:

1. Immediate, real-time results. Existing neuroscience hardware is expensive and complicated to use, and requires controlled environments during use. Consequently, both the data collection and analysis of data can be slow, at times taking weeks to get results. The immersion assessment system of the present disclosure enables second-by-second data capture that is processed in real-time, such that the analysis results may be obtained during and immediately after a given experience.

2. Measure reactions in any environment. While neuroscience laboratory settings enable strict control of the environment, labs are not where people live. In contrast, the immersion assessment system of the present disclosure enables data collection and analysis in situ, wherever the experience is taking place.

3. Outcome focused. The immersion assessment system of the present disclosure analyzes the heart rhythm data collected and aggregates the results in a way that provides actionable information supported by proven scientific research.

4. Remote data management. The immersion assessment system of the present disclosure enables remote collection of the necessary input data (i.e., heart rhythm) using wearable sensors connected to the immersion assessment system via, for example, wireless, cellular, or Bluetooth technology. Thus, any experience to be assessed may be monitored remotely.

5. Content control. Optionally, the immersion assessment system of the present disclosure enables real time control of the experience to be presented to participants as, for example, a live experience. For instance, based on real time assessment of the immersion level of participants, the content may be modified or different content be presented to participants.

6. Anonymity. The data collection (i.e., heart rhythm data input) may be aggregated and anonymized such that the participants can feel comfortable during assessments. That is, as participants are able to interact with neuroscience collection and neuroscience analytics using typical software-as-a-service (SaaS) privacy gates and levels of anonymity. Thus, partner companies and content creators may find it easier to recruit and encourage participation by a larger number of experience participants, and comply with consumer privacy and protection laws.

Furthermore, the immersion assessment system of the present disclosure may enable additional features such as online event management (including sending of event invitations, attendee registration, and event start/stop) and display of real-time immersion level to participants and/or stakeholders. The analysis results may be viewed in terms of, for example, a second-by-second line chart showing fluctuations in immersion levels throughout the experience, benchmarked metrics comparing the analysis results for a specific experience to existing norms, and secondary metrics, such as psychological safety, average experience scores, length and depth of deep immersion or emotional disconnect, and demographics of individuals most likely to engage with a particular experience.

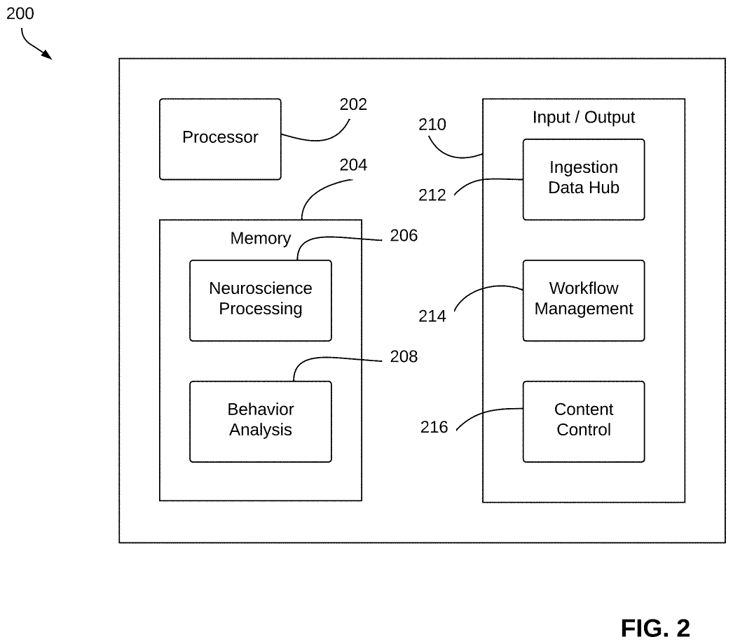

FIG. 2 is a block diagram of a computing system 200 for assessing the immersion level of one or more experience participants with presented content, in accordance with an embodiment. Computing system 200 may be a standalone computing system, in an example. Computing system 200 includes a processor 202 for controlling the operations of a memory 204, which includes programming such as neuroscience processing 206 (e.g., functions performed by neuroscience processing unit 130 of FIG. 1) and behavior analysis 208 (e.g., functions performed by behavior analysis unit 140 of FIG. 1), such programming being executable by processor 202. Processor 202 further controls an input/output interface 210, which is configured for receiving and transmitting data, such as heart rhythm data from one or more experience participants (e.g., experience participant 110 of FIG. 1), media content from content creators (e.g., media content 170 from content creators 162 of FIG. 1, and aggregated data and/or analysis history. In an example, input/output interface 210 includes an ingestion data hub 210, a workflow management unit 214, and a content control 216, which are analogous to ingestion data hub 120, workflow management unit 150, and content control unit 172 of FIG. 1. Thus, input/output interface may receive participant data and provide the received data to memory 204 to generate immersion analysis results, which then may be communicated outside of computing system 200.

As an alternative, portions of neuroscience processing, behavior analysis, data ingestion, workflow management, and/or content control may be performed outside of computing system 200, such as in the cloud (e.g., cloud 164 of FIG. 1), with input/output interface 210 controlling the flow of data between computing system 200 and the outside world.

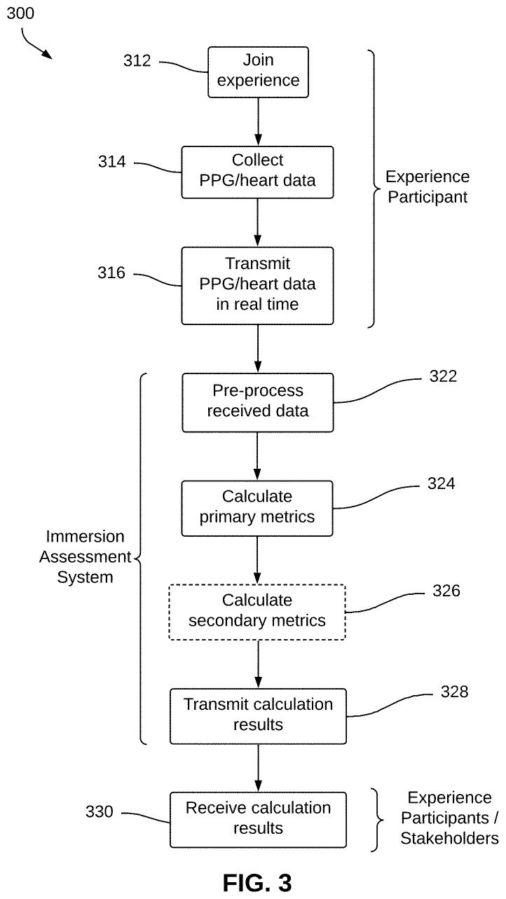

FIG. 3 shows a flow diagram for using a system for assessing the immersion level of an experience participant with presented content, in accordance with an embodiment. As shown in FIG. 3, in conjunction with FIG. 1, a process 300 begins when a participant (e.g., experience participant 110) opts to join an experience to be assessed (e.g., a live event or media content 170) in a step 312. Step 312 may include, for example, an opt-in agreement through an application interface or a manual signing of a waiver, in which the experience participant agrees to share physiological data (e.g., heart rhythm data) and in some cases demographic information collected during the experience. Then, in a step 314, heart rhythm data is collected from the participant, and the collected information is transmitted to the immersion assessment system (e.g., immersion assessment system 100). As shown in FIG. 3, steps 312, 314, and 316 are performed at devices controlled by the participant. It is noted that multiple participants may be performing steps 312, 314, and 316 at the same time to provide input data to the immersion assessment system.

Continuing to refer to FIG. 3, the heart rhythm data via PPG is received at the immersion assessment system to pre-process the received data in a step 322. Step 322 may be performed, for example, by ingestion data hub 120. Then primary metrics, such as immersion levels and psychological safety, are calculated in a step 324. Step 324 may be performed, for example, by neuroscience processing unit 130 by correlating the input data from participants with known neurophysiologic indicators, as described above. Optionally, secondary metrics may be calculated in a step 326 by, for example, behavior analysis unit 140 of FIG. 1. Such secondary metrics may include aggregated analyses of data from multiple participants and/or over the presentation time of the experience. The calculation results of the primary and/or secondary metrics are then transmitted to participants or stakeholders in a step 328, such as via application interface 114 and/or user interface aspects of workflow management unit 150 of the immersion assessment system 100. Finally, the calculation received by participants or other stakeholders (e.g., partner companies 160, content creators 162, or data aggregator in cloud server 164) in a step 330.

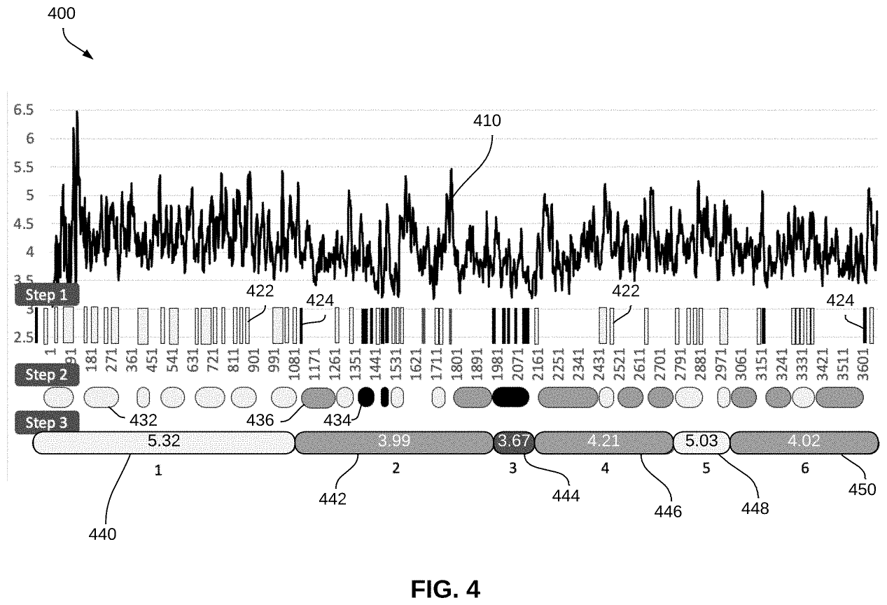

An example of data analysis that may be performed by immersion assessment system 100 is a clustering process to form what will be referred to as “pills” or clusters of time that segment an experience based on immersion scores. FIG. 4 shows an exemplary graph of processed heart rhythm data, along with an illustration of exemplary steps in analyzing the data presented in the graph, in accordance with an embodiment.

As described above, the immersion assessment system 100 receives heart rhythm data information via data capture mechanism 112 as raw data. This heart rhythm data information is processed by the various components within the immersion assessment system 100 such as ingestion data hub 120 and neuroscience processing unit 130 to provide a data graph 410 with the Y axis corresponding to calculated immersion value (normalized units) and the X axis corresponding to time (in seconds). That is, data graph 410 corresponds to the heart rhythm data that has been processed using established correlation between heart rhythms and measurable neurochemistry metrics (e.g., as discussed in the journal articles cited above) to provide a second-by-second immersion score.

Then, in a step 1, meaningful moments such as high immersion periods 422 (shown as light colored boxes), corresponding to peaks in the processed data graph 410, are identified. Similarly, low immersion periods 424 (shown as dark colored boxes), corresponding to low points in data graph 410, are identified.

In a step 2, trends in high immersion periods 422 and low immersion periods 424 are grouped into light “pills” (i.e., ovals) 432 and black pills 434. These groupings may be performed, for example, using thresholds involving the proximity of the identified high and low immersion periods, as well as the duration of each one of the high and low immersion periods. Additionally, neutral immersion periods (shown as gray pills), during which the experience participant is neither highly immersed or has low immersion are identified in lulls between the light or black pills.

Then, in a step 3, trends in the high, neutral, and low immersion periods are identified by grouping together adjacent pills. For instance, as shown in FIG. 4, a first time period 440 corresponds to relatively high immersion level by a participant being measured. Then the participant was neutral during a second time period 442, lost interest in a third time period 444, returned to neutral in a fourth time period 446, became highly immersed again in a fifth time period 448, then returned to neutral immersion level in a sixth time period 450. A numerical score (as shown within each time period 440, 442, 444, 446, 448, and 450) may be calculated for each of these time periods by averaging the calculated immersion levels within the total time encompassed by that time period.

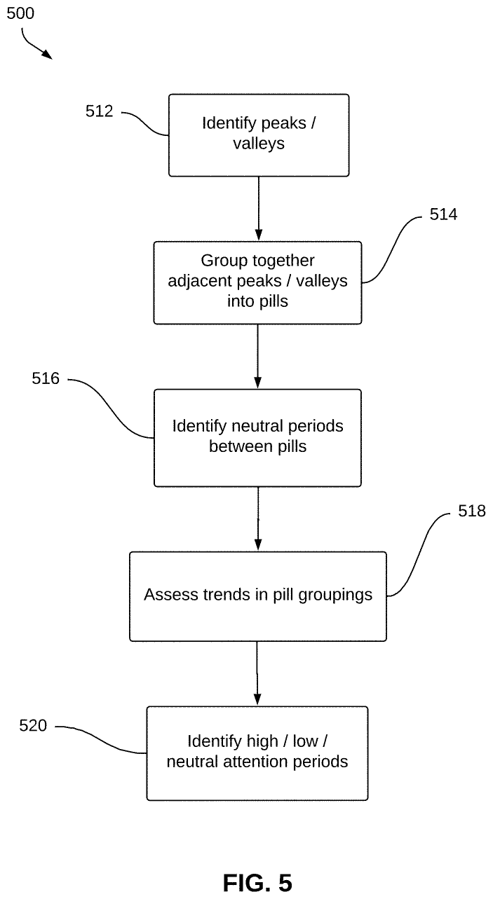

FIG. 5 shows a flow diagram for performing the steps corresponding to the illustration in FIG. 4, in accordance with an embodiment. As discussed relative to FIG. 4, a process 500 begins with a step 512 to identify the peaks and valleys in immersion, shown as the graph in FIG. 4. Adjacent peaks and valleys in immersion are grouped together into “pills” in a step 514. Further, neutral immersion periods between pills are identified in a step 516. Then, trends in the high/low/neutral pill groupings are assessed in a step 518, thus allowing identification of high/low/neutral immersion periods in a step 520.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel insights and advantages of this invention.

Accordingly, many different embodiments stem from the above description and the drawings. It will be understood that it would be unduly repetitious and obfuscating to literally describe and illustrate every combination and subcombination of these embodiments. As such, the present specification, including the drawings, shall be construed to constitute a complete written description of all combinations and subcombinations of the embodiments described herein, and of the manner and process of making and using them, and shall support claims to any such combination or subcombination.

As an example, while the described embodiments involve the use of PPG sensors to gather the data used in the immersion analysis, other types of heart activity sensors, such as electrocardiography (ECG) sensors, may be used. While commonly used PPG sensors in smartwatches and fitness monitors are certainly convenient for simultaneously gathering heart rhythm data from multiple participants at live events or group settings, participants in certain types of more passive experiences (e.g., movie or television viewing) may be monitored using more static means, such as ECG sensors that require each participant to be provided with multiple electrodes, or utilizing the built-in camera of smartphones that require each participant to press and hold a finger onto the surface of the camera lens.

Further, while aggregation of data from multiple participants in real time provide heretofore unavailable analytic capabilities related to immersion, certain embodiments described herein may be useful in situations involving one or a few participants.

In the specification, there have been disclosed embodiments of the invention and, although specific terms are employed, they are used in a generic and descriptive sense only and not for purposes of limitation. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the claimed invention

What is claimed is:

1. An immersion assessment system for assessing immersion levels of at least one experience participant based on heart rhythm data collected from the at least one participant during an experience, the system comprising:
an ingestion data hub for receiving and processing the heart rhythm data to provide clean data;
a neuroscience processing unit for analyzing the clean data and providing analytical results including primary metrics;
a behavior analysis unit for further analyzing the clean data and the analytical results to provide secondary metrics; and
a workflow management unit for controlling the ingestion data hub, the neuroscience processing unit, and the behavior analysis unit.

2. The immersion assessment system of claim 1, wherein the primary metrics includes at least one of immersion and psychological safety.

3. The immersion assessment system of claim 1, wherein the secondary metrics includes at least one of a comparison of the primary metrics with established norms, and identification of key moments during the experience.

4. The immersion assessment system of claim 1, further comprising:
a content control unit, interfaced with at least the neuroscience processing unit, for presenting the experience to at least one experience participant, and correlating the analysis results with specific parameters and timing associated with the experience.

5. The immersion assessment system of claim 1, further comprising an application interface, connected with the ingestion data hub, for receiving input from and providing an output to the at least one experience participant.

6. The immersion assessment system of claim 1, wherein the behavior analysis unit is configured for performing at least one of aggregate profiling, vertical analysis, and pattern analysis.

7. An immersion assessment system comprising:
a processor;
memory; and
an input/output interface,
wherein the input/output interface is configured for receiving heart rhythm data from at least one participant during an experience,
wherein memory includes programming executable by the processor to:
based on the heart rhythm data from the at least one participant, performing at least one of neuroscience processing and behavior analysis; and
generating an assessment of immersion levels of at least one participant during the experience.

8. The immersion assessment system of claim 7,
wherein the input/output interface is further configured for processing the heart rhythm data from the at least one participant to generate clean data, and providing the clean data to the memory.

9. The immersion assessment system of claim 7,
wherein the input/output interface is further configured for receiving media content for presentation to at least one participant in the experience, and
wherein the input/output interface is further configured for correlating the media content with the assessment of immersion levels of the at least one participant.

10. The immersion assessment system of claim 7,
wherein the memory further includes programming executable by the processor to perform at least one of aggregate profiling, vertical analysis, and pattern analysis based on the heart rhythm data received from the input/output interface.

11. The immersion assessment system of claim 7, wherein the input/output interface includes an input mechanism for receiving photoplethysmography (PPG) measurements of the at least one participant using a PPG device.

12. The immersion assessment system of claim 11, wherein the PPG device includes at least one of a smart watch, a fitness tracker, and a built-in camera of a smart device.
